(19) United States
(12) Patent Application Publication
(10) Pub. No.: US 2006/0177391 A1
(43) Pub. Date: Aug. 10, 2006

(54) Preparation, in particular cosmetic preparation, and the production and use thereof

(76) Inventors: Azra Swistowski, Nuremberg (DE); Christina Zech, Kaufering (DE)

Correspondence Address: BACHMAN & LAPOINTE, P.C., 900 CHAPEL STREET, SUITE 1201, NEW HAVEN, CT, 06510, US

(21) Appl. No.: 11/262,253

(22) Filed: Oct. 28, 2005

(30) Foreign Application Priority Data
Feb. 4, 2005 (DE) 10 2005 005 486.2

(51) Int. Cl. A61K 8/89

(52) U.S. Cl. 424/59; 424/63; 424/70.12; 424/70.7

(57) ABSTRACT

A preparation, in particular a cosmetic preparation, preferably in liquid or pasty form, for use on keratinic materials, in particular for decorating, coloring, improving the appearance of and caring for the skin and the skin appendages. The preparation is water-free and contains at least one wax with a dropping point above 60° C., a volatile oil, a non-volatile oil, an alkyl polysiloxane, an alcohol which is solid at ambient temperature, and a film-forming, flexible copolymer with a dropping point above 30° C.

### BACKGROUND OF THE INVENTION

The invention concerns a preparation, in particular a cosmetic preparation, preferably in liquid or pasty form, for use on the skin, on semi-mucous membranes, on mucous membranes and on keratinic materials such as hair, eyelashes and eyebrows, in particular for decorating, coloring and improving the appearance and for caring for the skin and the skin appendages. Preparations of that kind are used for example for makeup, in particular for the eyelashes and hair; such a preparation is then referred to as ‘mascara’. Basically the preparations according to the invention, with suitable adjustment and coloring, can also be used as makeup, concealer, camouflage, eyeshadow, eyeliner, lipliner, rouge, lip rouge, lip gloss, sun protection agent, sun block, temporary tattoo, colored effect sun protection for surfers and the like. Mascara preparations are known in the form of what is referred to as ‘block mascara’ or ‘cake mascara’, a solid form of preparation from which the material is removed by means of a moistened brush, in the form of ‘emulsion mascara’ in the form of O/W or W/O emulsions, or in the form of a solvent-based mascara. The use of a ‘block mascara’ is complicated if no water is available for moistening the brush or the material and another common name—namely ‘spit mascara’—shows that alternative forms of use can certainly entail serious microbiologically induced risks in terms of product and user.

Due to the type involved—at least one emulsifier must always be used in an ‘emulsion mascara’ in order to keep the continuous phase in intimate relationship with the dispersed phase. A disadvantage with such a product is generally that, after drying on the eyelashes, it can be dissolved again due to moisture from the environment, for example water, rain, perspiration and in particular the quite slightly alkaline tear fluid, whereby the durability of such preparations is reduced. The use of water-soluble polymers or polymers which can be dispersed in water also involves detrimental effects here. An example of such a polymer-bearing product is to be found in W Umbach, ‘Kosmetik’, Georg Thieme Verlag, Stuttgart, 1988, page 101. Other examples are to be found in the standard works in cosmetics literature, for example H Janistyn, ‘Handbuch der Kosmetika und Riechstoffe’, volume 3, Hathig-Verlag, 2nd edition, 1973, on pages 855-860 or G A Nowak, ‘Die kosmetischen Praparate’, Verlag Ziolkowsky, 3rd edition, 1984, pages 748-751. An emulsion mascara on a polymer base, which contains at least one wax and a pseudo-latex, should also be mentioned. A disadvantage with compositions of that kind is also that, after drying on eyelashes or hair, they form a brittle coating which can tear and flake off. In the case of a hair mascara, that effect can certainly be desired because such a preparation, in the form of temporary hair coloring, can later be easily removed again by brushing it out—if however fine particles flake off the eyelashes, they can land in the eye and result in mechanical irritation, or also color the region under the eye (what is referred to as the ‘panda bear effect’).

Solvent-bearing mascara preparations are also known, which contain only little or no water at all. Polar solvents such as ethanol, propan-1-ol, propan-2-ol, acetone, methylethylketone, ethylacetate, tetrahydrofuran, dichloromethane and the like automatically rule themselves out by virtue of their odor, their severely irritating effect on skin and mucous membranes and lack of environmental compatibility. Ethanol can at best be used in amounts below 15% by volume. Non-polar isoparaffins have admittedly already been used in the past—in regard to some thereof however, a point which tells against them is the aromatics content, which excludes them for cosmetic uses and the easy flammability of the low-boiling isoparaffins, whereby specific precautions are advised in the production of preparations of that kind. If however exclusively or predominantly higher-boiling C₁₁/C₁₂ isoparaffins are chosen, preparations which adhere very well to skin and hair are admittedly obtained, which have very good transfer resistance—however they then generally have very long drying times, which makes them markedly uncomfortable from the point of view of users. A further disadvantage is also that diffusion problems and stress crack corrosion can occur when using containers of given kinds of polypropylene.

Therefore the object of the invention was to provide a preparation in particular for coloring, improving the appearance of and caring for the skin and the skin appendages, which can be applied easily and uniformly to skin, semi-mucous membranes, mucous membranes, hair and eyelashes which has good coverage, which dries in a time considered by users as appropriate and as a result becomes wipe-resistant, which adheres for a long time, which does not migrate into the fine wrinkles in the skin and which is not transferred from the place of application on to other media such as for example cups, glasses, metal, textiles, or other areas of the skin and the like. That can give rise to unattractive colored impressions or films of fat, more specifically oily constituents which spread out well on the skin can migrate together with small amounts of pigment and leave unattractive traces behind on the skin. Products which do not adhere adequately to skin, semi-mucous membranes, mucous membranes, hair and eyelashes then have to be regularly re-applied.

### SUMMARY OF THE INVENTION

The foregoing object is obtained by providing a preparation, in particular a cosmetic preparation, preferably in liquid or pasty form, for use on keratinic materials, in particular for decorating, coloring, improving the appearance of and caring for the skin and the skin appendages. The preparation is water-free and contains at least one wax with a dropping point above 60° C., a volatile oil, a non-volatile oil, an alkyl polysiloxane, an alcohol which is solid at ambient temperature, and a film-forming, flexible copolymer with a dropping point above 30° C.

### DETAILED DESCRIPTION

The preparation which is preferably water-free and free of emulsifiers and which, optionally besides other waxes, contains at least one wax with a dropping point above 60° C., preferably above 70° C., at least one volatile oil, at least one non-volatile oil, at least one alkyl polysiloxane, at least one long-chain alcohol which is solid at ambient temperature (that is to say at about 22° C.) and at least one film-forming, flexible polymer or copolymer. In addition the preparation may also contain ingredients which are usual in cosmetics and which are permitted by the respective national legislation, solids such as additives and fillers, coloring agents, dispersing agents, light protection filters which are effective in the UV-A and UV-B range, anti-foam agents, preserving agents, antioxidants, anti-settlement agents, fragrance mixtures and the like. The preparation according to the invention is free of non-volatile mineral oils and triglycerides which are liquid or solid at ambient temperature.

The waxes are selected from waxes of animal, vegetable, mineral or synthetic origin or mixtures and hybrids thereof. The waxes used are of a hardness of between 2 and 40, wherein the hardness value is determined using the needle penetration method. The operation of determining the hardness value is effected in accordance with the American Standard ASTM D5: at a temperature of 25° C. a needle with a defined cone and weighing 2.5 g which is loaded with a weight of 47.5 g is caused to penetrate into a flat surface of a test body. The depth of penetration is determined in tenths of a millimeter after 5 seconds.

Beeswax, modified beeswax or so-called ‘cera bellina’, lanolin wax, Japan wax, candelilla wax, ouricuri wax, carnauba wax, rice wax, flower waxes or fruit waxes such as orange flower wax, orange wax, jasmine wax or apple wax, montan wax, microcrystalline wax, paraffin waxes, ozocerite, polyethylene waxes, waxes produced using the Fischer-Tropsch method, silicone waxes, long-chain esters such as for example cetyl palmitate, stearyl stearate, behenyl stearate, behenyl behenate, hydrated jojoba oil or mixtures thereof and their hybrids are preferably used. The amounts of such waxes or the mixtures thereof and/or their hybrids used are generally in the range of between 3 and 35% by weight, preferably between 5 and 25% by weight, and are particularly preferably between 8 and 20% by weight.

The volatile oils are selected from aromatics-free C₁₁/C₁₂ isoparaffins, straight-chain, branched or cyclic silicone oils such as for example hexamethyl disiloxane, octamethyl trisiloxane, decamethyl tetrasiloxane, dodecamethyl pentasiloxane, hexamethyl cyclotrisiloxane, octamethyl cyclotetrasiloxane, decamethyl cyclopentasiloxane, dodecamethyl cyclohexasiloxane or mixtures thereof. The amount of those volatile oils used is in the range of between 10 and 80% by weight, preferably between 15 and 65% by weight and quite particularly preferably between 25 and 50% by weight.

The non-volatile oils are selected from silicone oils such as dimethyl polysiloxane (dimethicone), phenyl trimethicone, diphenyl dimethicone, medium-chain or long-chain carboxylic acid esters such as jojoba oil, oleyl erucate, oleyl oleate, diethyl sebacate, hexyl laurate, dibutyl adipate, isostearyl isostearate or mixtures thereof. The amount of those non-volatile oils used is generally between 1 and 30% by weight and preferably between 5 and 15% by weight.

The alkyl dimethicones are selected from the dimethyl siloxanes of differing chain length, which have alkyl residues. The alkyl residues can have between 10 and 36 carbon atoms. They can also have hydroxyl groups and/or be joined by way of ether bridges with shorter alkyl chains. In the broadest sense esters of dimethiconols are to be classified among the alkyl dimethicones. Mention may be made here by way of example of substances such as lauryl dimethicone, cetyl dimethicone, cetearyl dimethicone, behenyl dimethicone, C24-28 alkyl dimethicone, bishydroxyethoxypropyl dimethicone, phenylpropylsilsesquisiloxane, dimethiconol stearate, hydroxypropyldimethicone behenate and mixtures thereof. In that respect the amount used is between 0.5 and 35% by weight, preferably between 1.5 and 25% by weight, quite particularly preferably between 5 and 15% by weight.

The long-chain alcohols are selected from the saturated, straight-chain or branched fatty alcohols which are solid at ambient temperature, of a chain length of between 14 and 48 carbon atoms, such as for example myristyl alcohol, cetyl alcohol, stearyl alcohol, behenyl alcohol, ceryl alcohol, myricyl alcohol or mixtures thereof. Preferably a mixture of alcohols of a chain length of between 20 and 40 carbon atoms is used. The amount of long-chain alcohols used in that case is between 0.5 and 20% by weight, preferably between 2.5 and 15% by weight, particularly preferably between 4 and 12% by weight.

An essential constituent of the preparation according to the invention is a long-chain linear dimethyl polysiloxane of a molecular weight of above 100,000 Daltons, preferably above 250,000 Daltons, quite particularly preferably above 450,000 Daltons.

To improve applicability, durability and transfer resistance, preferably a film-forming, flexible polymer or copolymer is used, having a dropping point of at least 30° C. Preferred in that respect is a long-chain alkyl/isoalkyl maleate/MA terpolymer. The amount thereof used is between 0.1 and 20% by weight, preferably between 0.5 and 12% by weight and quite particularly preferably between 2 and 8% by weight.

The fillers used are solids such as for example talcum, kaolin, healing earth, smectite, starch and modified starch, non-swelling starches, cellulose, polytetrafluoroethylene powder (Teflon), polyamide powder (Nylon), mica, insoluble metal soaps such as Mg stearate, Ca stearate, Sr stearate or Zn stearate and/or inorganic or organic pigments and lakes of organic coloring agents. The following may be mentioned by way of example of the latter: titanium dioxide, zinc oxide, yellow, red or black iron oxide, chrome oxide green, hydrated chrome oxide green, ultramarine, Berlin Blue (Ferric Blue), manganese violet, carmine, pearlescent pigments, for example micas coated with titanium dioxide and colored micas coated with titanium dioxide and metal oxides, bismuth oxychloride, bismuth oxychloride coated with metal oxides, flake-form metal powders of aluminum, bronze, brass, copper, silver and gold, as well as lakes of organic dyes with aluminum, barium, calcium, strontium or zirconium or mixtures of the specified substances. That list is only by way of example and is in no way definitive. Those additions are effected with the proviso that they are also allowed by the respective national or regional legislation. The amounts used in that respect are within the limits of the maximum amounts regulated by the respective cosmetic legislation. The quantitative proportions of fillers and coloring agents are in that connection in a range of between 0.01 and 70% by weight, preferably in a range of between 1 and 55% by weight and quite particularly preferably in a range of between 5 and 35% by weight. The high level of sheen which can be achieved and the high level of color intensity, caused by the refractive index of the silicone used, is advantageous when employing the coloring agents set forth hereinbefore by way of example, in water-free preparations. The preparations are independent of any influences caused by pH-values, for example the development of hydrogen sulfide when using manganese violet or the possible liberation of hydrogen when using aluminum powders is also not to be expected because of the absent water as a reaction partner. When using silica, surface-coated, preferably hydrophobised products are advantageously used.

In sun protection agents, in particular so-called ‘sun blocks’, besides oil-soluble light filter substances which are regulated by the respective cosmetic regulations, such as for example isoamyl or ethylhexyl-p-methoxy cinnamate or 4-methylbenzylidene camphor, it is also possible to use combinations with metal oxides such as for example titanium dioxide, cerium oxide, zinc oxide and zirconium oxide in the form of so-called ‘nanopigments’, of mean particle sizes in the range of between 5 and 25 nm.

For the purposes of viscosity regulation and stabilisation of the preparation according to the invention, it is certainly also possible to use per se known additives such as bentonites, hectorites, montmorillonites or also organically modified derivatives of those additives and their combinations, which then possibly have to be activated in a suitable manner known to the man skilled in the relevant art. As is known, small additions of diethyl carbonate, propylene carbonate or alcohol solutions of a-hydroxy acids are suitable for that purpose. Naturally it is also possible to produce oleogels using 12-hydroxystearic acid and behenates, for example behenamidopropyl dimethylamine behenate—the corresponding processes are known to the man skilled in the relevant art for example from DE 102 01 370 C2.

As the preparations according to the invention are preferably water-free, it is also possible to dispense with the use of preserving agents and antioxidants if stressing tests carried out previously provide indications in that respect.

The preparations according to the invention are present in viscous form or in the form of workable pastes and can be easily and uniformly distributed on the skin, on semi-mucous membranes, on mucous membranes and on hair and eyelashes. The viscosity of the preparations according to the invention is adjusted according to the desired purpose of use and is in the range of between 50 and 1500 mPas in the case of the liquid to viscous preparations and in the range of between 1.5 and 500 Pas in the case of the pasty preparations. In that respect the viscosity is determined in conventional manner with a commercially available viscosimeter at a rotary speed of 1 rpm with a plate/ball or plate/plate measuring device, in the case of the latter with a gap width of 0.4 mm. After drying, the preparations according to the invention form at the place where they are applied an elastic layer which is perceived as being pleasant and which does not move, which does not run, which does not migrate and which can remain at the place at which it is applied for the entire day. It can be removed again in a manner which is known to the users—by suitable makeup removal agents or cloths, by means of a cleaning lotion or by washing off with fine soap or with corresponding mild tenside preparations. It can be filled in known manner into containers suitable for the desired purpose of use such as bottles with applicators contained therein (spatula, fine brush, coarse brush, and the like), jars, tubes, pots or tubs (bowls) and can be taken therefrom again by the user. However, because of the improved hygiene conditions that this involves, they can also be introduced into suitable applicator devices, referred to as ‘dispenser mechanisms’, and applied therefrom. So-called pressure-actuated metering dispensers with a trailing piston which subsequently moves along with the material are basically known. The possibility of filling a preparation in a container in the form of a dual-chamber aerosol is also known. Applicator devices as are known for example from U.S. Pat. No. 6,238,117 or U.S. Pat. No. 6,309,128 are suitable for the application of small amounts as are required for example in the lip and eye region as they allow very attractive fine metering.

If the preparations according to the invention are produced as ‘mascara preparations’ for coloring the eyelashes, then they are intended not only to impart color to the eyelashes but also to strengthen them, in order to cause them to appear visually thicker and stronger and to give them curl. The preparations are then usually colored black, brown, blue and dark grey. If the intention is only to make the natural color of eyelashes stronger and more impressive, the recommendation is for the mascara preparation to be produced in transparent form without fillers and coloring agents. If the intention is to achieve particular color effects under long-wave UV-light (so-called ‘black light’), in order for example to achieve particular effects under disco lighting, it is possible to add to such a mascara preparation titanium dioxide in the form of nanopigments or stilbene derivatives which are allowed for cosmetics use. The current fashion is for the tips of the eyelashes to be accentuated with contrasting colors, for example light green, moss-green, yellow, orange or red. Preferably the above-mentioned inorganic pigments which are allowed for the region of the eyes are suitable for that purpose, as coloring agents, but also for example lakes of organic dyes such as FD&C Blue No 1 (C.I.-No 42090), FD&C Red No 40 (C.I.-No 16035), or FD&C Yellow No 50 (C.I.-No 19140). If the preparation according to the invention is to be used as hair mascara, the color selection is naturally more versatile as in that case then it is also possible to use lakes of organic dyes which are only allowed for external use. If preparations are wanted which give particular color effects under disco lighting, it is possible to use the fluorescent dyes which are suitable for that purpose and which are sufficiently known to the man skilled in the relevant art—see in that respect for example WO 2004/108108. It is also possible to use fine glitter particles of material-colored polymer carriers such as polyesters, polycarbonates or the like, in effect preparations. Mascara preparations which contain fine fibers, for example Nylon, silk, cellulose, polyester, Teflon, Rayon and the like have already long been usual for strengthening and lengthening eyelashes.

Containers for mascara are commercially available and are sufficiently known to the man skilled in the art—he only has to consider here that the selected brush provides for good wetting of the eyelashes and in so doing separates them and that the brush and the scraper portion and additionally the container and the mascara preparation are well matched to each other. If a mascara preparation and an effect mascara are offered together, then a packaging which can be combined from two individual units is recommended—even those combination packagings in which one unit can be provided with a smaller volume and a smaller brush have in the meantime become commercially available. As normally a person has more hair on the head than eyelashes, a larger container naturally has to be adopted for hair mascara.

The preparation according to the invention will now be described in detail by means of the following Examples which however do not definitively describe it. In that respect the amounts are given in percent by weight, in each case with respect to the total weight of the respective preparation. In an individual case the amounts involved can be slightly greater than or less than the specified amounts, in which case nonetheless the preparations according to the invention are produced. That is in no way unexpected to the man skilled in the relevant art, in consideration of the widely available great variety of suitable components of such preparations, so that the man skilled in the art knows that, when the amounts used exceed or are less than the specified amounts in that way, this does not constitute a departure from the range of the present invention. The INCI names which are known to the man skilled in the relevant art are used to identify the raw materials involved.

### EXAMPLE 1

### Pasty Mascara Preparation

| | |
|---|---|
| Octamethyl trisiloxane | 35.900 |
| Dimethicone (mw 500,000) | 17.500 |
| Cera microcristallina (81-84° C.) | 12.000 |
| C24-28 alkyldimethicone | 8.000 |
| Candelilla cera | 7.000 |
| C30-38 olefin/isopropyl maleate/MA copolymer | 6.500 |
| C20-C40 alcohols | 2.500 |
| Black iron oxide (C.I. No 77.499) | 8.000 |
| Silica | 2.000 |
| Tocopherol acetate | 0.350 |
| Fragrance | 0.250 |

For production purposes the raw materials of items Nos 2-6 are melted at about 80° C., then the coloring agent (black iron oxide) and the silica are added and the mixture is then passed three times through a three-roll mill. The mixture is now transferred into a vacuum process installation, octamethyl trisiloxane, tocopherol acetate and the fragrance mixture are added thereto, the mixture is thoroughly mixed, deaerated under a slight vacuum, cooled to about 28-30° C. and transferred into storage containers.

For the production of further colors, instead of just the black iron oxide, mixtures of black, red and yellow iron oxide are used to produce a brown mascara preparation, mixtures of black iron oxide with titanium dioxide are used to produce a gray mascara preparation and ultramarine, possibly also using titanium dioxide, is used for the production of a blue mascara preparation. Effect colors can be produced by the use of yellow and/or red iron oxide (yellow, orange, red mascara preparation), chrome oxide green (moss-green mascara preparation), hydrated chrome oxide green (turquoise mascara preparation), possibly also in each case mixed with titanium dioxide. In that respect it is a matter for the discretion of the man skilled in the art to effect variations in the foregoing Example, according to the desired requirements in terms of color shade and color intensity. In that respect reductions in the amount of pigment can be compensated by an increase in the proportion of volatile oils or by the addition of uncoated mica or talcum.

### EXAMPLE 2

### Pasty, Colorless Mascara Preparation

| | |
|---|---|
| Decamethyl tetrasiloxane | 41.600 |
| Dimethicone (mw 350,000) | 12.500 |
| Cera microcristallina (81-84° C.) | 12.000 |
| C24-28 alkyldimethicone | 5.500 |
| Carnauba wax (Copernicia Cerifera) | 6.500 |
| C30-38 olefin/isopropyl maleate/MA copolymer | 4.500 |
| C20-C40 alcohols | 4.500 |
| Titanium dioxide (nanopigment 10-20 nm) | 8.000 |
| Silica | 2.500 |
| Silica silylate | 1.800 |
| Tocopherol acetate | 0.350 |
| Fragrance | 0.250 |

The production of Example 2 is effected in a similar manner to Example 1. This mascara preparation produces on the eyelash an almost colorless and transparent coating which thickens and slightly lengthens the eyelash. Under long-wave UV-light that preparation exhibits an intensive, light-blue lighting effect. In order to obtain a product which is entirely colorless even under UV-light, the titanium dioxide nanopigment is replaced by the corresponding amount of decamethyl tetrasiloxane.

### EXAMPLE 3

### Pasty Hair Mascara

| | |
|---|---|
| Decamethyl tetrasiloxane | 37.900 |
| Dimethicone (mw 300,000) | 14.500 |
| Cera microcristallina (81-84° C.) | 12.000 |
| C24-28 alkyldimethicone | 5.500 |
| Beeswax, white | 6.500 |
| C30-38 olefin/isopropyl maleate/MA copolymer | 4.500 |
| C20-C40 alcohols | 4.500 |
| Titanium dioxide | 2.500 |
| Red iron oxide (C.I.-No 77.491) | 5.000 |
| Yellow iron oxide (C.I.-No 77.492) | 1.500 |
| Polyester-3, Red 28 *) | 5.000 |
| Tocopherol acetate | 0.350 |
| Fragrance | 0.250 |

*) ‘Polyester-3, Red 28’ is a solid solution of D&C Red No 28 (C.I.-No 45410) in a polyester matrix.

The production of Example 3 is similar to Example 1. That mascara preparation is particularly suitable for the temporary coloring of individual strands and produces on the hair an orange-yellow coloring which under long-wave UV-light exhibits a bright red fluorescence.

### EXAMPLE 4

### Pasty Effect Sun Protection for Surfers

| | |
|---|---|
| Octamethyl trisiloxane | 40.900 |
| Dimethicone (mw 500,000) | 8.500 |
| Cera microcristallina (81-84° C.) | 5.000 |
| C24-28 alkyldimethicone | 6.500 |
| Beeswax, white | 5.500 |
| C30-38 olefin/isopropyl maleate/MA copolymer | 4.500 |
| C20-C40 alcohols | 3.500 |
| Isoamyl p-methoxycinnamate | 4.000 |
| 4-Methylbenzylidene camphor | 3.500 |
| Buxus chinensis (jojoba oil) | 3.000 |
| Titanium dioxide (nanopigment 10-20 nm) | 8.000 |
| Titanium dioxide (C.I.-No 77.891) | 1.500 |
| Ultramarine (C.I.-No 77.007) | 5.000 |
| Tocopherol acetate | 0.350 |
| Fragrance | 0.250 |

The production of Example 4 is effected similarly to Example 1. The result obtained is a radiantly blue paste with the effect of a sun block. If color variants are desired, the ultramarine can also be replaced by yellow or red iron oxide, hydrated chrome oxide green, chrome oxide green, manganese violet, titanium dioxide or mixtures thereof. Applied by means of a roll-on applicator, it is possible to apply lines or drawings to the skin.

### EXAMPLE 5

### Pasty, Colorless Sun Block

| | |
|---|---|
| Octamethyl trisiloxane | 41.400 |
| Dimethicone (mw 500,000) | 9.500 |
| Cera microcristallina (81-84° C.) | 7.000 |
| C24-28 alkyldimethicone | 6.500 |
| Ouricuri wax | 4.500 |
| C30-38 olefin/isopropyl maleate/MA copolymer | 4.500 |
| C20-C40 alcohols | 3.500 |
| Isoamyl p-methoxycinnamate | 5.500 |
| 4-Methylbenzylidene camphor | 4.500 |
| Buxus chinensis (jojoba oil) | 3.000 |
| Titanium dioxide (nanopigment 10-20 nm) | 8.000 |
| Quaternium-18 hectorite | 1.500 |
| Tocopherol acetate | 0.350 |
| Fragrance | 0.250 |

The production of Example 5 is effected similarly to Example 1. The result obtained is a colorless translucent paste which is suitable for filling in tubes. It affords a high level of light protection in the region around SF 25-30.

### EXAMPLE 6

### Pasty Eyeshadow

| | |
|---|---|
| Octamethyl cyclotetrasiloxane | 35.000 |
| Decamethyl cyclopentasiloxane | 14.000 |
| Dimethicone (mw 500,000) | 3.600 |
| Ouricuri wax | 6.200 |
| C20-C40 alcohols | 4.800 |
| C30-38 olefin/isopropyl maleate/MA copolymer | 5.200 |
| Hydroxypropyl dimethicone behenate | 7.200 |
| Lauryl dimethicone | 7.200 |
| Silica | 2.000 |
| Titanated mica (C.I.-No 77891) | 8.000 |
| Mica | 2.000 |
| Hydrated chrome oxide green (C.I.-No 77289) | 3.200 |
| Ultramarine (C.I.-No 77007) | 0.800 |
| Tocopherol | 0.600 |
| Fragrance | 0.200 |

The production of Example 6 is effected similarly to Example 1, but the pearl pigments are added only after the second pass through the three-roll mill. The powerfully turquoise material with a bright pearl sheen is suitable for filling into small pots, or for improved handling and product hygiene, an elongate container with an applicator fixed to a stem, a so-called ‘deer foot applicator’, as a holder, which is connected to the closure cap.

### EXAMPLE 7

### Fluid Foundation, Water-Resistant

| | |
|---|---|
| Octamethyl cyclotetrasiloxane | 34.200 |
| Decamethyl cyclopentasiloxane | 42.700 |
| Dimethiconol stearate | 0.850 |
| Bishydroxyethoxypropyl dimethicone | 5.100 |
| Cetearyl dimethicone/vinyl dimethicone cross-polymer | 1.700 |
| Cera microcristallina (81-84° C.) | 2.200 |
| Candelilla cera | 0.900 |
| C20-28 alkyl dimethicone | 1.700 |
| C20-C40 alcohols | 1.700 |
| C30-38 olefin/isopropyl maleate/MA copolymer | 1.700 |
| Tocopherol | 0.550 |
| Silica | 0.450 |
| Titanium dioxide (C.I.-No 77891) | 3.500 |
| Yellow iron oxide (C.I.-No 77492) | 1.200 |
| Red iron oxide (C.I.-No 77491) | 0.850 |
| Black iron oxide (C.I.-No 77499) | 0.450 |
| Fragrance | 0.250 |

The production of Example 7 is effected similarly to Example 1. The result obtained is a light-brown pasty preparation which has good coverage and is long-lasting, with good transfer resistance and which is highly resistant to moisture, for example perspiration. It is highly suitable for being filled into tubes.

### EXAMPLE 8

### Fluid Lipliner

| | |
|---|---|
| Octamethyl cyclotetrasiloxane | 68.000 |
| Cera microcristallina | 6.800 |
| Candelilla cera | 1.400 |
| Dimethicone (mw 500,000) | 4.100 |
| C24-28 alkyl dimethicone | 2.700 |
| C20-C40 alcohols | 0.700 |
| Tocopherol | 0.500 |
| Octadecene/MA copolymer | 0.700 |
| Diethyl sebacate | 0.900 |
| Silica | 0.400 |
| Titanium dioxide (C.I.-No 77891) | 4.100 |
| Red iron oxide (C.I.-No 77491) | 6.000 |
| FD&C Red No 40 (C.I.-No 16035) | 3.500 |
| Fragrance | 0.200 |

The production of Example 8 is effected similarly to Example 1, in which respect, in order to obtain a material which can be processed on a three-roll mill, about 20-25% of the volatile silicone oil is added to the hot material. The result obtained is a powerfully red, still fluid preparation which affords good coverage and which can be applied in small containers by means of a fiber tip fitted into the closure cap. The preparation is transfer-resistant and does not migrate into the fine wrinkles of the skin.

### EXAMPLE 9

### Fluid Eyeliner

| | |
|---|---|
| Octamethyl trisiloxane | 52.000 |
| Octamethyl cyclotetrasiloxane | 20.000 |
| Cera microcristallina | 5.000 |
| Rice bran wax | 2.000 |
| C20-C40 alcohols | 1.600 |
| Dimethicone (mw 500,000) | 2.100 |
| C30-38 olefin/isopropyl maleate/MA copolymer) | 1.800 |
| Phenylpropylpolysilsesquisiloxane | 2.400 |
| Tocopherol | 0.400 |
| Silica | 0.500 |
| Black iron oxide (C.I.-No 77499) | 10.000 |
| Ultramarine (C.I.-No 77007) | 1.300 |
| Carmine (C.I.-No 75470) | 0.700 |
| Fragrance | 0.200 |

The production of Example 9 is effected similarly to Example 8. The result obtained is a fluid preparation of a deep black color and of good durability and transfer resistance. For ease of handling, it is filled into the containers referred to in Example 8.

### EXAMPLE 10

### Pasty Covering Cream for Couperose, Water-Resistant

| | |
|---|---|
| Octamethyl trisiloxane | 9.200 |
| Octamethyl cyclotetrasiloxane | 46.000 |
| Decamethyl cyclopentasiloxane | 15.200 |
| Cera microcristallina | 3.800 |
| C24-28 alkyldimethicone | 5.700 |
| C20-C40 alcohols | 1.900 |
| C30-38 olefin/isopropyl maleate/MA copolymer) | 1.900 |
| Methoxy amodimethicone silsesquioxane copolymer | 1.900 |
| Dimethicone (mw 500,000) | 4.700 |
| Tocopherol | 0.450 |
| Mica | 1.000 |
| Titanium dioxide (C.I.-No 77891) | 4.500 |
| Hydrated chrome oxide green (C.I.-No 77269) | 3.500 |
| Fragrance | 0.250 |

The production of Example 10 is effected similarly to Example 1. The result obtained is a light-turquoise preparation with good coverage and very good transfer resistance, which can be easily distributed uniformly on the skin. The preparation is suitable for filling in tubes. It serves to conceal skin anomalies such as pigment spots and small visible blood vessels (couperose and rosacea), by virtue of its complementary color. After that preparation dries on the skin, a suitable makeup can be applied according to the taste of the respective wearer.

1. A preparation in liquid or pasty form, which is free of emulsifiers, comprises a mixture comprising:
a) at least one wax with a dropping point above 60° C.,
b) at least one volatile oil,
c) at least one non-volatile oil,
d) at least one alkyl polysiloxane,
e) at least one long-chain alcohol which is solid at ambient temperature, and
f) at least one film-forming, flexible polymer or copolymer with a dropping point of at least 30° C.

2. A preparation as set forth in claim 1, wherein the mixture is water-free.

3. A preparation as set forth in claim 1, wherein the mixture is free of non-volatile mineral oils and triglycerides which are liquid or solid at ambient temperature.

4. A preparation as set forth in claim 1, wherein the mixture is a cosmetic preparation for use on the skin, on semi-mucous membranes, on mucous membranes, on hair, on eyelashes and/or on eyebrows.

5. A preparation as set forth in claim 4, wherein the mixture is used for decoration, coloring, improving appearance and caring.

6. A preparation as set forth in claim 1, wherein the wax is selected from the group consisting of vegetable, animal, mineral, synthetic origin and mixtures thereof.

7. A preparation as set forth in claim 6, wherein the wax has a needle penetration in accordance with ASTM D5 of between 2 and 40, determined at a temperature of 25° C. and with a penetration time of 5 sec, with a total weight of the test body of 50 g.

8. A preparation as set forth in claim 1, wherein the wax is selected from the group consisting of beeswax, modified beeswax (so-called ‘cera bellina’), candelilla wax, carnauba wax, ouricuri wax, rice wax, montan wax, microcrystalline wax, paraffin wax, ozocerite, polyethylene wax, wax produced in accordance with the Fischer-Tropsch process, silicone wax and mixtures thereof.

9. A preparation as set forth in claim 8, wherein the preparation comprises a further wax and/or long-chain esters which are selected from the group consisting of lanolin wax, Japan wax, orange flower wax, orange wax, cetyl palmitate, stearyl stearate, behenyl stearate, behenyl behenate, hydrated jojoba oil, and mixtures thereof.

10. A preparation as set forth in claim 1, wherein the amount of the wax is in the range of between 3 and 35% by weight.

11. A preparation as set forth in claim 1, wherein the amount of the wax is in the range of between 5 and 25% by weight.

12. A preparation as set forth in claim 1, wherein the amount of the wax is in the range of between 8 and 20% by weight.

13. A preparation as set forth in claim 1, wherein the volatile oil is selected from the group consisting of aromatics-free C₁₁/C₁₂ isoparaffins, linear, branched or cyclic silicone oils, and mixtures thereof.

14. A preparation as set forth in claim 13, wherein silicone oil is selected from the group consisting of hexamethyl disiloxane, octamethyl trisiloxane, decamethyl tetrasiloxane, dodecamethyl pentasiloxane, hexamethyl cyclotrisiloxane, octamethyl cyclotetrasiloxane, decamethyl cyclopentasiloxane, dodecamethyl cyclohexasiloxane, and mixtures thereof.

15. A preparation as set forth in claim 13, wherein the amount of volatile oil is between 10 and 80% by weight.

16. A preparation as set forth in claim 13, wherein the amount of volatile oil is between 15 and 65% by weight.

17. A preparation as set forth in claim 13, wherein the amount of volatile oil is between 25 and 50% by weight.

18. A preparation as set forth in claim 1, wherein the non-volatile oil is selected from the group consisting of silicone oils, medium-chain and long-chain carboxylic acid esters, and mixtures thereof.

19. A preparation as set forth in claim 18, wherein the non-volatile silicone oils are selected from the group consisting of dimethyl polysiloxane (dimethicone), phenyl trimethicone, diphenyl dimethicone, and mixtures thereof.

20. A preparation as set forth in claim 19, wherein the dimethyl polysiloxane is selected from the group consisting of long-chain linear dimethyl polysiloxanes of a molecular weight above 100,000 Daltons.

21. A preparation as set forth in claim 19, wherein the dimethyl polysiloxane is selected from the group consisting of long-chain linear dimethyl polysiloxanes of a molecular weight above 250,000 Daltons.

22. A preparation as set forth in claim 19, wherein the dimethyl polysiloxane is selected from the group consisting of long-chain linear dimethyl polysiloxanes of a molecular weight above 450,000 Daltons.

23. A preparation as set forth in claim 18, wherein the medium-chain and long-chain carboxylic acid esters are selected from the group consisting of jojoba oil, oleyl erucate, oleyl oleate, hexyl laurate, dibutyl adipate, diethyl sebacate, isostearyl isostearate, and mixtures thereof.

24. A preparation as set forth in claim 1, wherein the amount of the non-volatile oils is between 1 and 30% by weight.

25. A preparation as set forth in claim 1, wherein the amount of the non-volatile oils is between 5 and 15% by weight.

26. A preparation as set forth in claim 1, wherein the alkyl polysiloxane comprises alkyl dimethicone selected from the group consisting of lauryl dimethicone, cetyl dimethicone, cetearyl dimethicone, behenyl dimethicone, C24-28 alkyl dimethicone, bishydroxyethoxy propyl dimethicone, phenyl propyl silsesquisiloxane, dimethiconol stearate, hydroxypropyl dimethicone behenate and mixtures thereof.

27. A preparation as set forth in claim 26, wherein the amount of alkyl dimethicones used is between 0.5 and 35% by weight.

28. A preparation as set forth in claim 26, wherein the amount of alkyl dimethicones used is between 1.5 and 25% by weight.

29. A preparation as set forth in claim 26, wherein the amount of alkyl dimethicones used is between 5 and 15% by weight.

30. A preparation as set forth in claim 1, wherein the long-chain alcohol which is solid at ambient temperature is selected from the group consisting of saturated straight-chain or branched-chain alcohols of a chain length of between 14 and 48 carbon atoms, and mixtures thereof.

31. A preparation as set forth in claim 27, wherein the long-chain alcohol which is solid at ambient temperature is selected from the group consisting of myristyl alcohol, cetyl alcohol, stearyl alcohol, behenyl alcohol, ceryl alcohol, myricyl alcohol, and mixtures thereof.

32. A preparation as set forth in claim 27, wherein the long-chain alcohol which is solid at ambient temperature is selected from the group consisting of a mixture of alcohols of a chain length of between 20 and 40 carbon atoms.

33. A preparation as set forth in claim 30, wherein the amount of the alcohols used is between 0.5 and 20% by weight.

34. A preparation as set forth in claim 30, wherein the amount of the alcohols used is between 2.5 and 15% by weight.

35. A preparation as set forth in claim 30, wherein the amount of the alcohols used is between 4 and 12% by weight.

36. A preparation as set forth in claim 1, further comprising a film-forming, flexible polymer or copolymer having a dropping point of at least 30° C.

37. A preparation as set forth in claim 35, wherein the film-forming, flexible polymer or copolymer is selected from the group consisting of C30-38 olefin/isopropyl maleate/MA copolymer, cetearyl dimethicone/vinyl dimethicone cross-polymer, octadecene/MA copolymer, methoxy amodimethicone silsesquioxane copolymer, and mixtures thereof.

38. A preparation as set forth in claim 36, wherein the amount of film-forming flexible polymer or copolymer is between 0.1 and 20% by weight.

39. A preparation as set forth in claim 36, wherein the amount of film-forming flexible polymer or copolymer is between 0.5 and 12% by weight.

40. A preparation as set forth in claim 1, wherein the mixture further comprises additives selected from the group consisting of thickening agents, light filter substances, preserving agents, antioxidants, dispersing additives, anti-settlement additives, fragrance mixtures, fillers, solids, coloring agents, and mixtures thereof.

41. A preparation as set forth in claim 40, wherein the coloring agents are selected from the group consisting of inorganic pigments, organic pigments, lakes of organic dyes, and mixtures thereof.

42. A preparation as set forth in claim 41, wherein the amount of coloring agents is between 0.01 and 70% by weight.

43. A preparation as set forth in claim 41, wherein the amount of coloring agents is between 1 and 55% by weight.

44. A preparation as set forth in claim 41, wherein the amount of coloring agents is between 5 and 35% by weight.

45. A preparation as set forth in claim 1, wherein the mixture is selected from the group consisting of makeup, concealer, camouflage, eyeshadow, eyeliner, lipliner, rouge, lip rouge, lip gloss, sun protection agent, sun block, temporary tattoo, mascara, hair mascara, sun protection, and covering cream for couperose and rosacea.
